(19) United States
(12) Patent Application Publication
(10) Pub. No.: US 2019/0293643 A1
(43) Pub. Date: Sep. 26, 2019

(54) Method for Predicting the Response of a Subject with an Autoimmune Disease to a B Cell-Targeting Therapy

(71) Applicant: UCL Business plc, London (GB)

(72) Inventor: Geraldine Cambridge, London (GB)

(21) Appl. No.: 16/348,744

(22) PCT Filed: Nov. 9, 2017
(86) PCT No.: PCT/GB2017/053373

§ 371 Date: May 9, 2019

(30) Foreign Application Priority Data
Nov. 11, 2016 (GB) 1619074.6

(51) Int. Cl. G01N 33/564; C07K 16/28

(52) U.S. Cl. CPC G01N 33/564; C07K 16/2851; G01N 2800/52; G01N 2333/70596; G01N 2800/104; C07K 2317/24

(57) ABSTRACT

The present invention relates to a method for predicting the response of a subject with an autoimmune disease to a B cell-targeting therapy. In particular, the invention relates to predicting the response of a patient by analysing the level of soluble CD23 (sCD23) and B cell activation factor (BAFF) in a sample from said subject. The B cell-targeting therapy can be an anti-CD20 antibody. The autoimmune disease can be systemic lupus erythematosus (SLE). Methods of treatment, compositions and kits are disclosed.

### FIELD OF THE INVENTION

The present invention relates to a method for predicting the response of a subject with an autoimmune disease to a B cell-targeting therapy. In particular, the invention relates to predicting the response of a patient by analysing the level of soluble CD23 and B cell activation factor (BAFF) in a sample from said subject.

### BACKGROUND

Autoimmune diseases result from an abnormal or pathological immune response to self tissue.

Autoimmunity is the presence of a self-reactive immune response (e.g., auto-antibodies or self-reactive T-cells), with or without damage or pathology resulting from it. This may be restricted to certain organs (e.g. in autoimmune thyroiditis) or involve a particular tissue in different places (e.g. Goodpasture's disease which may affect the basement membrane in both the lung and the kidney).

Approximately 24 million (7%) of people in the United States are affected by an autoimmune disease. Women are more commonly affected than men, and autoimmune diseases often start during adulthood. The first autoimmune diseases were described in the early 1900s.

Various therapies are presently available for the treatment of autoimmune diseases. Nonsteroidal anti-inflammatory drugs (NSAIDs) and immunosuppressants are often used. Immunoglobulins may also be used.

Treatment plans and clinical outcomes depend on the type and severity of the condition.

Existing therapies can have variable clinical outcomes in different patients.

For example, in the case of B cell-targeting therapies, interruption of B cell maturation and differentiation associated with removal of B cells underlie clinical success, but the site of B cell abnormality underlying autoimmunity may differ between patients.

Predictive response markers would be useful in identifying subjects who may be predicted to have an improved response to a particular therapy, for example a B cell-targeting therapy, and hence the subject may be suitable to receive the B cell-targeting therapy. Such markers would also be useful to identify subjects who may not be suitable for treatment with the B cell-targeting therapy, i.e. those that may be predicted to have a decreased or poorer response to B cell-targeting therapies.

There is, therefore, a need in the art for suitable predictive response markers which would enable methods for identifying subjects who will respond well to B cell-targeting therapies, thereby facilitating stratification of subjects.

The present inventor has found that the levels of soluble CD23 (also referred to herein as sCD23) and BAFF in a subject can be predictive of the response of said subject to a B cell-targeting therapy.

### BRIEF DESCRIPTION OF THE FIGURES

FIG. 1: Summary data for 39 consecutive patients with a diagnosis of SLE (ACR Guidelines) referred to UCLH, and from whom at least 4 serial serum samples were available within a 24 month period. All patients were Rituximab naïve. Times between samples varied and therefore data was grouped in terms of order of sample collection which was at least 2-3 months apart in each patient. Samples were tested for sCD23 and BAFF. Data were grouped for each respective time point (A being first, B being second etc). p values were calculated using non-parametric statistics (Wilcoxon test for paired samples) in order to assess relative change between variables.

FIG. 2: The same data is represented as in FIG. 1 but results for individual patients are shown.

### SUMMARY OF THE INVENTION

The present inventor has identified that soluble CD23 and BAFF are biomarkers for predicting response of a subject to a B cell-targeting therapy.

As such, in one aspect the invention provides a method for predicting whether a subject with an autoimmune disease will respond to a B cell-targeting therapy, wherein said method comprises determining the level of soluble CD23 and BAFF in a sample from said subject.

The invention therefore encompasses a method for identifying a subject with an autoimmune disease who is suitable or not suitable for treatment with a B cell-targeting therapy, wherein said method comprises determining the level of soluble CD23 and BAFF in a sample from said subject.

In a further aspect the invention provides the use of soluble CD23 and BAFF as biomarkers for predicting whether a subject with an autoimmune disease will respond to a B cell-targeting therapy, and hence for identifying a subject with an autoimmune disease who is suitable or not suitable for treatment with a B cell-targeting therapy.

Another aspect of the present invention relates to a method for predicting the response of a subject with an autoimmune disease to a B cell-targeting therapy, comprising the steps of:

a) measuring a level of soluble CD23 and BAFF in a sample pre-obtained from the subject to obtain a value or values representing this level; and
b) comparing the value or values from step a) to a standard value or set of standard values.

The measuring of a level of soluble CD23 and BAFF may be performed in vitro or ex vivo in a sample or samples pre-obtained from the subject. Pre-obtained refers to the fact that the sample is obtained before it is subjected to any method involving measuring the level of the biomarker.

The response which is predicted may be improved or poorer (decreased) response to said B cell-targeting therapy.

In one aspect, the methods and uses of the invention only require analysis of soluble CD23 and BAFF as biomarkers (i.e. in one aspect the methods and uses of the invention do not comprise determining levels of any biomarkers other than soluble CD23 and BAFF). Although, for the avoidance of doubt, the levels of the biomarkers may be standardised by comparison to one or more housekeeping genes, proteins or markers, as described herein.

In another aspect, the present invention provides a kit for predicting whether a subject with an autoimmune disease will respond to a B cell-targeting therapy, wherein the kit comprises one or more agent capable of determining the level of soluble CD23 and BAFF in a sample obtained from the subject; wherein the kit optionally comprises a set of instructions.

In yet another aspect, the present invention provides a composition comprising a therapeutically effective amount of a B cell-targeting therapy for use in the treatment of an autoimmune disease in a subject predicted to respond to a B cell-targeting therapy (i.e. be suitable for treatment with a B cell-targeting therapy) by the method of the present invention.

In a further aspect, the invention provides a method of treating an autoimmune disease in a subject, wherein said method comprises the following steps:
i) identifying a subject with an autoimmune disease who is suitable for treatment with a B cell-targeting therapy according to the method of the invention; and
ii) treating said subject with a B cell-targeting therapy.

The invention also provides a B cell-targeting therapy for use in a method of treating an autoimmune disease in a subject, the method comprising:
i) identifying a subject with an autoimmune disease who is suitable for treatment with a B cell-targeting therapy according to the method of the invention; and
ii) treating said subject with a B cell-targeting therapy.

In still another aspect, the present invention provides a method of treating an autoimmune disease in a subject predicted to respond to a B cell-targeting therapy by the method of invention (i.e. be suitable for treatment with a B cell-targeting therapy), comprising administering a therapeutically effective amount of a B cell-targeting therapy to the subject.

In a further aspect, the invention relates to a method of treating an autoimmune disease, in a subject in need thereof, comprising measuring the level of soluble CD23 and BAFF in a sample from the subject to obtain a value or values representing this level, and treating the subject with a B cell-targeting therapy, if the level of soluble CD23 and BAFF in said sample is higher or lower than a standard value or set of standard values, as described herein.

### DETAILED DESCRIPTION

### Autoimmune Diseases

As discussed herein, autoimmune diseases encompass diseases in which there is a pathological immune response to self tissues.

Autoimmune diseases include, for example, acute idiopathic thrombocytopenic purpura, chronic idiopathic thrombocytopenic purpura, dermatomyositis, Sydenham's chorea, myasthenia gravis, systemic lupus erythematosus, lupus nephritis, rheumatic fever, polyglandular syndromes, bullous pemphigoid, diabetes mellitus, Henoch-Schonlein purpura, post-streptococcal nephritis, erythema nodosurn, Takayasu's arteritis, Addison's disease, rheumatoid arthritis, multiple sclerosis, sarcoidosis, ulcerative colitis, erythema multiforme, IgA nephropathy, polyarteritis nodosa, ankylosing spondylitis, Goodpasture's syndrome, thromboangitis ubiterans, Sjogren's syndrome, primary biliary cirrhosis, Hashimoto's thyroiditis, thyrotoxicosis, scleroderma, chronic active hepatitis, polymyositis/dermatomyositis, polychondritis, pemphigus vulgaris, Wegener's granulomatosis, membranous nephropathy, amyotrophic lateral sclerosis, tabes dorsalis, giant cell arteritis/polymyalgia, pernicious anemia, rapidly progressive glomerulonephritis, psoriasis, fibrosing alveolitis, and vasculitis (including anti-neutrophil cytoplasmic antibody (ANCA)-associated vasculitis).

Some examples of autoimmune diseases are discussed further below.

In rheumatoid arthritis the immune system produces antibodies that attach to the linings of joints. Immune system cells then attack the joints, causing inflammation, swelling, and pain. If untreated, rheumatoid arthritis causes gradually causes permanent joint damage. Treatments for rheumatoid arthritis can include various oral or injectable medications that reduce excessive activity of the immune system.

Systemic lupus erythematosus (lupus). Subjects with lupus develop autoimmune antibodies that can attach to tissues throughout the body. The joints, lungs, blood cells, nerves, and kidneys are commonly affected in lupus. Treatment often requires daily oral prednisone, a steroid that reduces immune system function.

Inflammatory bowel disease (IBD). The immune system attacks the lining of the intestines, causing episodes of diarrhea, rectal bleeding, urgent bowel movements, abdominal pain, fever, and weight loss. Ulcerative colitis and Crohn's disease are the two major forms of IBD. Oral and injected immune-suppressing medicines can treat IBD.

Multiple sclerosis (MS). The immune system attacks nerve cells, causing symptoms that can include pain, blindness, weakness, poor coordination, and muscle spasms. Various medicines that suppress the immune system can be used to treat multiple sclerosis.

Type 1 diabetes mellitus. Immune system antibodies attack and destroy insulin-producing cells in the pancreas. By young adulthood, people with type 1 diabetes may require insulin injections to survive.

Guillain-Barre syndrome. The immune system attacks the nerves controlling muscles in the legs and sometimes the arms and upper body. Weakness results, which can sometimes be severe. Filtering the blood with a procedure called plasmapheresis is the main treatment for Guillain-Barre syndrome.

Chronic inflammatory demyelinating polyneuropathy. Similar to Guillian-Barre, the immune system also attacks the nerves in CIDP, but symptoms last much longer. About 30% of patients can become confined to a wheelchair if not diagnosed and treated early. Treatment for CIDP and GBS are essentially the same.

Psoriasis. In psoriasis, overactive immune system blood cells called T-cells collect in the skin. The immune system activity stimulates skin cells to reproduce rapidly, producing silvery, scaly plaques on the skin.

Graves' disease. The immune system produces antibodies that stimulate the thyroid gland to release excess amounts of thyroid hormone into the blood (hyperthyroidism). Symptoms of Graves' disease can include bulging eyes as well as weight loss, nervousness, irritability, rapid heart rate, weakness, and brittle hair. Destruction or removal of the thyroid gland, using medicines or surgery, is usually required to treat Graves' disease.

Hashimoto's thyroiditis. Antibodies produced by the immune system attack the thyroid gland, slowly destroying the cells that produce thyroid hormone. Low levels of thyroid hormone develop (hypothyroidism), usually over months to years. Symptoms include fatigue, constipation, weight gain, depression, dry skin, and sensitivity to cold. Taking a daily oral synthetic thyroid hormone pill restores normal body functions.

Myasthenia gravis. Antibodies bind to nerves and make them unable to stimulate muscles properly. Weakness that gets worse with activity is the main symptom of myasthenia gravis. Mestinon (pyridostigmine) is the main medicine used to treat myasthenia gravis.

Vasculitis. The immune system attacks and damages blood vessels in this group of autoimmune diseases. Vasculitis can affect any organ, so symptoms vary widely and can occur almost anywhere in the body. Treatment includes reducing immune system activity, usually with prednisone or another corticosteroid.

The present invention encompasses subjects with any autoimmune disease, such as (but not limited to) those discussed herein.

In one aspect of the invention as described herein the autoimmune disease is systemic lupus erythematosus (SLE).

As mentioned above, SLE is an autoimmune disease in which the body's immune system mistakenly attacks healthy tissue in many parts of the body. Symptoms vary between people and may be mild to severe. Common symptoms include painful and swollen joints, fever, chest pain, hair loss, mouth ulcers, swollen lymph nodes, feeling tired, and a red rash which is most commonly on the face. Often there are periods of illness (flares), and periods of remission when there are few symptoms.

### Biomarker

Methods and uses according to the present invention comprise determining the level of biomarkers, namely soluble CD23 and BAFF.

CD23 (Fc epsilon RII, or FccRII) is the “low-affinity” receptor for IgE, the antibody isotype involved in allergy and resistance to parasites, and is important in regulation of IgE levels. CD23 is a C-type lectin and is found on mature B cells, activated macrophages, eosinophils, follicular dendritic cells, and platelets. Soluble CD23 fragments (sCD23) of sizes ranging from 16 to 37 kDa are released from the cell surface and further processed via cleavage by metalloproteinases (principally ADAM10) and cysteine proteases. All fragments contain the C-terminal lectin domain, and the 25 kDa sCD23 fragment predominates in serum. The reference to “soluble” CD23 as used herein is intended to mean any CD23 molecule or fragment thereof that is found in soluble form (i.e. released from the cell surface).

BAFF is also known as B Lymphocyte Stimulator (BLyS) and TNF- and APOL-related leukocyte expressed ligand (TALL-1) and the Dendritic cell-derived TNF-like molecule (CD257 antigen; cluster of differentiation 257). BAFF is a cytokine that belongs to the tumour necrosis factor (TNF) ligand family. This cytokine is a ligand for receptors TNFRSF13B/TACI, TNFRSF17/BCMA, and TNFRSF13C/BAFF-R. This cytokine is expressed in B cell lineage cells.

Amino acid and nucleotide sequences of human soluble CD23 and BAFF are available from publicly accessible databases, e.g. under the accession numbers as shown in Table 1 below

| | CD23 | BAFF |
|---|---|---|
| Entrez | 2208 | 10673 |
| Ensembl | ENSG00000104921 | ENSG00000102524 |
| UniProt | P06734 | Q9Y275 |
| RefSeq | NC_000019.10 | NC_000013.11 |
| (DNA) | NC_018930.2 | NC_018924.2 |
| RefSeq | NP_001193948.2 | NP_001139117.1 |
| (protein) | NP_001207429.1 | NP_006564.1 |
| | NP_001993.2 | |

Amino acid and nucleotide sequences corresponding to further variants and homologs of the above genes, as well as genes found in other species, may be found in similar publicly accessible databases or by identifying sequences showing homology to the above human sequences.

The biomarker of the invention may be a protein or nucleic acid. The nucleic acid may be a ribonucleic acid (RNA) or deoxyribonucleic acid (DNA). The RNA may be a pre-mRNA or mRNA.

In a preferred embodiment, the biomarker is a protein or polypeptide.

Thus, in one embodiment, the method or use of the invention as described herein comprises determining the protein or polypeptide level of soluble CD23 and BAFF.

### Biomarker Level

The method of the present invention comprises the step of determining a level of soluble CD23 and BAFF in a sample from a subject.

The method according to the present invention may further comprise the step of comparing the level of said biomarkers in the sample to a reference value, wherein the level of biomarkers in the sample compared to the reference value is predictive of the response to B cell-targeting therapy in the subject.

By “determining a level” it is meant measuring—either quantitatively or semi-quantitatively—the amount of a particular substance. Typically, the determination will reveal the absolute level of a substance in a sample from a subject, or the level of a substance relative to the level of a reference sample or value.

A level of a substance may be determined more than once in a given sample, for example for the purpose of statistical calculations. Alternatively or in addition, a level may be determined one or more times in more than one sample obtained from a subject.

In a preferred embodiment, the level of the biomarker of the invention is determined in the form of protein or polypeptide.

In one embodiment, the level of protein or polypeptide biomarker of the invention is measured or determined relative to the level of a reference protein or polypeptide biomarker value.

Applicable techniques for determining the level of a biomarker in accordance with the present invention are known to the person skilled in the art.

In a preferred embodiment, the level of the biomarker of the invention is determined by detecting protein (polypeptide) levels.

The step of determining the level of the biomarker of the invention may involve detection of the polypeptide using a technique such as flow cytometry, antibody-based arrays, enzyme linked immunosorbent assay (ELISA), non-antibody protein scaffolds (e.g. fibronectin scaffolds), radioimmuno-assay (MA), western blotting, aptamers or mass spectrometry for example. Such techniques are routine in the art.

In a preferred embodiment of the invention as described herein, the protein (polypeptide) level is determined by ELISA.

An ELISA may be performed according to general methods which are known in the art. For example, the ELISA may be a sandwich or competitive ELISA. ELISA kits suitable for determining levels of soluble CD23 and BAFF are commercially available, for example from R&D Systems.

Various enzyme-substrate labels are available for use with such ELISAs, e.g. as disclosed in U.S. Pat. No. 4,275,149. The enzyme generally catalyses a chemical alteration of the chromogenic substrate that can be detected. For example, the enzyme may catalyse a colour change in a substrate, or may alter the fluorescence or chemiluminescence of the substrate. Examples of enzymatic labels include peroxidase such as horseradish peroxidase (HRPO), alkaline phosphatase, beta-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are well known.

In one aspect of the invention the level of the biomarkers is determined in the form of a nucleic acid, such as an mRNA transcript.

Suitable techniques for determining the level of nucleic acid are known in the art. Such techniques include, but are not limited to, Northern blot analysis, nuclease protection assays (NPA) e.g. RNAse protection assays, reverse transcriptase-PCR (RT-PCT), quantitiative PCR (qPCR), array, microarray, DNA microchip, DNA sequencing including mini-sequencing, primer extension, hybridization with allele-specific oligonucleotides (ASO), oligonucleotide ligation assays (OLA), PCR using allele-specific primers (ARMS), dot blot analysis, flap probe cleavage approaches, restriction fragment length polymorphism (RFLP), kinetic PCR, and PCR-SSCP, in situ hybridisation, fluorescent in situ hybridisation (FISH), pulsed field gel electrophoresis (PFGE) analysis, Southern blot analysis, single stranded conformation analysis (SSCA), denaturing gradient gel electrophoresis (DGGE), temperature gradient gel electrophoresis (TGGE), denaturing HPLC (DHPLC), and combinations of the above.

By “higher level” or “increased level”, it is meant that the relative or absolute level of the biomarker is of a substantially higher value compared to a reference value.

By “lower level” or “decreased level” it is meant that the relative or absolute level of the biomarker is of a substantially lower value compared to a reference value.

For example, a “higher” level may be taken as higher than a normal level, and a “lower” level may be taken as lower than a normal level.

For example, according to the “Quantikine ELISA” for human BAFF and soluble CD23 available from R&D Systems, the mean for sCD23 in serum from healthy controls (n=36) is 2515±1004 pg/ml; range 1235-5025, and for BAFF the mean in healthy controls is 850±465 pg/ml; range 584-1186.

In one aspect, therefore, a high or higher level of sCD23 may be taken as more than about 5000 pg/ml, for example more than about 5100, 5200, 5300, 5400, 5500, 5600, 5700, 5800, 5900, or 6000 pg/ml. In one aspect a high or higher level of sCD23 is more than 5025 pg/ml.

Conversely, a low or lower level of sCD23 may be taken as less than about 5000 pg/ml, for example less than about 4900, 4800, 4700, 4600, 4500, 4400, 4300, 4200, 4100, 4000, 3500, 3000, 2500, 2000, 1500, 1000 or 500 pg/ml. In one aspect a low or lower level of sCD23 is less than 5025 pg/ml.

In one aspect of the invention, a high or higher level of BAFF may be taken as more than about 1000 pg/ml, for example more than about 1100, 1200, 1300, 1400, 1500, 1600, 1700, 1800, 1900, or 2000 pg/ml. In one aspect a high or higher level of BAFF is more than 2000 pg/ml.

Conversely, a low or lower level of BAFF may be taken as less than about 2000 pg/ml, for example less than about 1900, 1800, 1700, 1600, 1500, 1400, 1300, 1200, 1100, 1000 or 500 pg/ml. In one aspect a low or lower level of BAFF is less than 2000 pg/ml.

As discussed in the present Examples, subjects may be classified as having “higher” or “lower” levels of soluble CD23 and BAFF using the upper limit of normal (ULN) as the cut-off point.

One skilled in the art will be able to determine, in the context of the invention, what constitutes a suitable higher or lower level of soluble CD23 and BAFF, and could carry out the appropriate analysis accordingly.

The higher or lower levels may be determined within the context of a cohort of subjects.

A “higher” level may correspond to a number greater than the median level in a cohort. A “lower” level may correspond to a number less than the median level in a cohort.

The level may be determined in relation to a cut-off or reference value.

In embodiments of the present invention, preferably the level of biomarker in the test sample differs by at least 1%, at least 5%, at least 10%, at least 20%, at least 30%, or at least 50% compared to the reference value.

### Reference Value

In certain embodiments of the present invention, biomarker levels are compared to reference values.

“Reference values” include but are not limited to, values obtained from reference subjects (and samples obtained therefrom), or pre-determined absolute values.

Typically, a reference value is derived from a subject who has an autoimmune disease, a subject known to be suffering from a particular autoimmune disease, or a subject who has or is receiving treatment for an autoimmune disease.

In the context of the present invention, the reference value may be derived from one or more of:
(a) a normal subject who does not have an autoimmune disease
(b) a subject who has an autoimmune disease;
(c) a subject known to be suffering from a particular autoimmune disease;
(d) a subject who has or is receiving treatment for an autoimmune disease;
(e) a subject who is refractory to one or more treatments for an autoimmune disease.

The reference value may be, for example, a predetermined measurement of a level of soluble CD23 and BAFF which is present in a sample from a normal subject.

The reference value may, for example, be based on a mean or median level of the biomarker in a control population of subjects, e.g. 5, 10, 100, 1000 or more subjects (who may either be age- and/or gender-matched or unmatched to the test subject) who show no symptoms of an autoimmune disease.

The reference value may be determined using corresponding methods to the determination of biomarker level in the test sample, e.g. using one or more samples taken from a control population of subjects. For instance, in some embodiments biomarker levels in reference value samples may be determined in parallel assays to the test samples.

In alternative embodiments, the reference value may have been previously determined, or may be calculated or extrapolated, without having to perform a corresponding determination on a reference value with respect to each test sample obtained.

In a preferred embodiment, the reference value is a range of values. For example, it may be determined that normal subjects present levels of a biomarker of the invention within a particular range. Equally, subjects suffering from an autoimmune disease may present levels of a biomarker of the invention within a particular “disease” range. Reference values, and in particular ranges of values may be optimised over time as more data is obtained and analysed.

In one aspect of the present invention, a higher level of soluble CD23 in combination with a lower level of BAFF in the sample from the subject compared to a reference subject is indicative of a predicted improved response to a B cell-targeting therapy.

In a further aspect of the invention, a higher level of BAFF in combination with a lower level of soluble CD23 in the sample from the subject compared to a reference subject is indicative of a predicted improved response to a B cell-targeting therapy.

In another aspect, a lower level of BAFF in combination with a lower level of soluble CD23 in the sample from the subject compared to a reference subject is indicative of a predicted decreased or poorer response to a B cell-targeting therapy. In one aspect the B cell-targeting therapy is rituximab.

In another aspect, a higher level of BAFF in combination with a higher level of soluble CD23 in the sample from the subject compared to a reference subject is indicative of a predicted decreased or poorer response to a B cell-targeting therapy.

In one aspect, the improved response to a B cell-targeting therapy may be an increase in B cell depletion. The major goal of B cell depletion therapy is to destroy malignant B lineage cells or autoimmune disease producing B cells in patients with cancers or autoimmune diseases, while at the same time retaining protective B cell immunity. Thus, the present invention facilitates the identification of subjects who may have an improved or better response in terms of B cell depletion following a B cell-targeting therapy.

In one aspect the improved response to a B cell-targeting therapy may be an increase in BILAG response.

“BILAG” stands for British Isles Lupus Activity Group. The BILAG is an organ-specific 86-question assessment based on the principle of the doctor's intent to treat, which requires an assessment of improved (1), the same (2), worse (3), or new (4) over the last month. Within each organ system, multiple manifestations and laboratory tests are combined into a single score for that organ. The resulting scores for each organ can be A through E, where A is very active disease, B is moderate activity, C is mild stable disease, D is resolved activity, and E indicates the organ was never involved. There are eight general headings; General, Mouth and Skin, Neurological, Joints and Muscles, Cardiovascular and Pulmonary, Blood Vessel Inflammation (Vasculitis), Kidney, and Blood.

In one aspect of the invention, the level of the biomarker of the invention and/or the reference value is standardised by comparison to one or more housekeeping genes, proteins or markers.

Levels of housekeeping genes, proteins or markers are known in the art not to fluctuate in response to varying experimental conditions. Suitable housekeeping genes are known in the art and are described in Silver N., et al (BMC Mol Biol. 2006 Oct. 6; 7:33; “Selection of housekeeping genes for gene expression studies in human reticulocytes using real-time PCR”)

Suitable examples include, but are not limited to, GAPDH (glyceraldehyde 3-phosphate dehydrogenase), β-actin, SDHA (succinate dehydrogenase), HPRT1 (hypoxanthine phosphoribosyl transferase 1), HBS1L (HBS1-like protein), AHSP (alpha haemoglobin stabilising protein) and B2M (beta-2-microglobulin). GAPDH is particularly preferred. The skilled person would appreciate that any housekeeping gene, protein or marker could be used for the purposes of the invention.

### Kit

The present invention provides a kit for predicting whether a subject with an autoimmune disease will respond to a B cell-targeting therapy, wherein the kit comprises one or more agents capable of determining the level of soluble CD23 and BAFF in a sample obtained from the subject; wherein the kit optionally comprises a set of instructions.

In yet a further aspect, the present invention provides the use of a kit of the invention for predicting whether a subject with an autoimmune disease will respond to a B cell-targeting therapy.

The kit may therefore be used to identify subjects suitable for treatment with a B cell-targeting therapy.

### Sample

The sample may be or may be derived from a biological sample, such as a blood sample, urine sample, cheek swab, a biopsy specimen, a tissue extract, an organ culture or any other tissue or cell preparation from a subject.

In theory, the presence of a protein biomarker according to the present invention can be determined by extracting protein from any tissue of the body.

The sample may be or may be derived from an ex vivo sample.

Preferably, the sample is, or is derived from blood, in particular peripheral blood. In a preferred embodiment the sample is a serum sample.

Preferably, the sample is, or is derived from, whole blood or a fraction of whole blood.

In embodiments wherein the biomarker of the invention is a polypeptide the sample may be, or may be derived from, blood cells.

In one aspect of the method or use of the present invention as discussed herein, the method or use comprises the step of obtaining a sample as discussed herein from the subject. Suitable methods of obtaining a sample will be known to one skilled in the art.

In one aspect the step of obtaining a sample comprises obtaining a blood sample, preferably a blood serum sample.

### Subject

In a preferred embodiment of the present invention, the subject is a mammal, preferably a cat, dog, horse, donkey, sheep, pig, goat, cow, mouse, rat, rabbit or guinea pig, but most preferably the subject is a human.

The subject may be any age, gender or ethnicity.

In one aspect the subject is Caucasian.

The subject may show one or more signs or symptoms of an autoimmune disease. The subject may have been previously characterised as having an autoimmune disease, and may previously have received treatment for an autoimmune disease. The method of the present invention may be used to stratify subjects for treatment with a B cell targeting therapy.

As defined herein “treatment” refers to reducing, alleviating or eliminating one or more symptoms of the disease, disorder or infection which is being treated, relative to the symptoms prior to treatment.

“Prevention” (or prophylaxis) refers to delaying or preventing the onset of the symptoms of the disease, disorder or infection. Prevention may be absolute (such that no disease occurs) or may be effective only in some individuals or for a limited amount of time.

### B Cell-Targeting Therapy

There are a number of therapies or therapeutic interventions that target B cells (B cell depletion therapy).

Current strategies include targeting B-cell surface antigens, cytokines that promote B-cell growth and functions, and B- and T-cell interactions.

Several antibodies have been developed as B cell-targeting therapies.

Anti-CD20 antibodies target CD20 on B cells. CD20 belongs to the tetraspan family of integral membrane proteins. Its function is largely unknown but it may be the case that this B-cell differentiation antigen may play a central role in T-cell-independent antibody responses. Anti-CD20 B-cell depletion was initially explored for the treatment of B-cell malignancies. Rituximab, a chimeric antibody comprising the mouse Fab and the human IgG1κFc portion of an anti-CD20 monoclonal antibody, was first marketed and approved by the FDA to treat B-cell lymphomas and showed significant clinical benefits. Rituximab achieves B-cell depletion through a number of different mechanisms, including direct induction of programmed cell death, antibody-dependent cellular cytotoxicity and complement-dependent cytotoxicity. Newer generations of anti-CD20 biologics, including the fully human MAb ofatumumab and other engineered versions with improved antibody-dependent cellular cytotoxicity killing, are in clinical development.

In addition, Ocrelizumab is a humanized monoclonal antibody against CD20.

Anti-CD22 and CD19 antibodies also target B cells. Based on a similar rationale to target B-cell-specific surface antigens that are expressed broadly across different B-cell subsets, therapeutics against CD22 and CD19 are also being explored. CD22, a member of the sialoadhesin subclass of the Ig superfamily, is a lectin receptor that binds α2,6-linked sialic acid residues. The tyrosine-based inhibition motifs in the CD22 signalling domain confer its negative regulatory effect on BCR, CD19/21 and CD45 signalling. As such, it plays a role in BCR-induced cell death and B-cell survival in the periphery. In contrast, CD19 is a transmembrane protein that is physically associated with BCR and functions to potentiate BCR signalling.

Epratuzumab is a humanized anti-CD22 MAb.

BL22 is also an anti-CD22 Mab. This antibody is fused with Pseudomonas aeruginosa exotoxin A, is undergoing clinical trials for the treatment of B-cell malignancies. A similar version of anti-CD19 with a maytansinoid conjugate is also under consideration.

There are also therapies that target cytokines to limit B cell growth and functions. Such therapies include anti-BAFF antibodies, such as belimumab.

Other B cell-targeting therapies target IL6 signalling, or the CD40/CD40 ligand interaction.

The method and use of the present invention may also have utility in predicting the response of a subject with an autoimmune disease to agents that target components of the alpha-interferon pathway, i.e. alpha-interferon targeting agents. The method and use of the present invention may also have utility in predicting the response of a subject with an autoimmune disease to agents that target Toll-like receptors (TLRs), and TLR signalling.

### Rituximab

In a preferred aspect of the invention as described herein, the method or use is useful in predicting the response of a subject to rituximab, that is to say the B cell-targeting therapy is rituximab.

Rituximab destroys both normal and malignant B cells that have CD20 on their surface and is therefore used to treat diseases which are characterized by having too many B cells, overactive B cells, or dysfunctional B cells.

Rituximab is used in the treatment of rheumatoid arthritis. Rituximab is also widely used off-label to treat difficult cases of multiple sclerosis, systemic lupus erythematosus, chronic inflammatory demyelinating polyneuropathy and autoimmune anaemias. It is also used in the management of kidney transplant recipients.

In one aspect of the present invention the autoimmune disease is systemic lupus erythematosus and the B cell-targeting therapy is rituximab.

All embodiments and optional features described herein apply equally to all aspects of the invention as described herein.

### Variants, Derivatives, Analogues, Homologues and Fragments

In addition to the specific proteins and nucleotides mentioned herein, the invention also encompasses the use of variants, derivatives, analogues, homologues and fragments thereof.

In the context of the invention, a variant of any given sequence is a sequence in which the specific sequence of residues (whether amino acid or nucleic acid residues) has been modified in such a manner that the polypeptide or polynucleotide in question substantially retains its function. A variant sequence can be obtained by addition, deletion, substitution, modification, replacement and/or variation of at least one residue present in the naturally-occurring protein or polynucleotide.

The term “derivative” as used herein, in relation to proteins or polypeptides of the invention includes any substitution of, variation of, modification of, replacement of, deletion of and/or addition of one (or more) amino acid residues from or to the sequence providing that the resultant protein or polypeptide substantially retains at least one of its endogenous functions.

The term “analogue” as used herein, in relation to polypeptides or polynucleotides includes any mimetic, that is, a chemical compound that possesses at least one of the endogenous functions of the polypeptides or polynucleotides which it mimics.

Typically, amino acid substitutions may be made, for example from 1, 2 or 3 to 10 or 20 substitutions provided that the modified sequence substantially retains the required activity or ability. Amino acid substitutions may include the use of non-naturally occurring analogues.

Proteins used in the invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R H |
| AROMATIC | | F W Y |

The term “homologue” as used herein means an entity having a certain homology with the wild type amino acid sequence and the wild type nucleotide sequence. The term “homology” can be equated with “identity”.

A homologous sequence may include an amino acid sequence which may be at least 50%, 55%, 60%, 65%, 70%, 75%, 80%, 85% or 90% identical, preferably at least 95% or 97% or 99% identical to the subject sequence. Typically, the homologues will comprise the same active sites etc. as the subject amino acid sequence. Although homology can also be considered in terms of similarity (i.e. amino acid residues having similar chemical properties/functions), in the context of the invention it is preferred to express homology in terms of sequence identity.

A homologous sequence may include a nucleotide sequence which may be at least 50%, 55%, 60%, 65%, 70%, 75%, 80%, 85% or 90% identical, preferably at least 95% or 97% or 99% identical to the subject sequence. Although homology can also be considered in terms of similarity, in the context of the invention it is preferred to express homology in terms of sequence identity.

Homology comparisons can be conducted by eye or, more usually, with the aid of readily available sequence comparison programs. These commercially available computer programs can calculate percentage homology or identity between two or more sequences.

Percentage homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the nucleotide sequence may cause the following codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percentage homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al. (1984) Nucleic Acids Res. 12: 387). Examples of other software that can perform sequence comparisons include, but are not limited to, the BLAST package (see Ausubel et al. (1999) ibid—Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, called BLAST 2 Sequences is also available for comparing protein and nucleotide sequences (see FEMS Microbiol. Lett. (1999) 174: 247-50; FEMS Microbiol. Lett. (1999) 177: 187-8).

Although the final percent homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62.

Once the software has produced an optimal alignment, it is possible to calculate percent homology, preferably percent sequence identity. The software typically does this as part of the sequence comparison and generates a numerical result.

“Fragments” of a full length protein or polynucleotide are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleotide.

Various modifications and variations of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be covered by the present invention.

### EXAMPLES

### Example 1

### Methods

98 serum samples from 26 SLE patients (ACR 1982 revised criteria) were taken 3 to 13 months prior to their first rituximab (RTX) treatment. BAFF and soluble CD23 were determined using ELISA [upper limits of normal (ULN) given by manufacturers (R&D Systems): soluble CD23>5000 pg/ml; BAFF>2 ng/ml]

Results were analyzed in relation to B cell depletion (CD19+B cells<5/μl), clinical characteristics, anti-dsDNA and British Institute of BILAG response at 6 months.

### Results

Table 2 shows clinical data and results.

TABLE 2
Characteristics and clinical responses to initial treatment with Rituximab in 26 patients with Lupus:
Relationships with different patterns of serum sCD23 and BAFF before treatment

| | Group I | | | | |
|---|---|---|---|---|---|
| | High sCD23, | Group II | Group III | Group IV | |
| | Low BAFF | High sCD23, High BAFF | Low sCD23, Low BAFF | Low sCD23, High BAFF | Total |
| | n = 3 | n = 8 | n = 6 | n = 7 | n = 26 |
| Ethnicity | | | | | |
| Caucasian | 5 (100%) | 3 (37.5%) | 3 (56%) | 3 (42.9%) | 14 (53.8%) |
| Afro-Caridium | 0 (0%) | 3 (62.5%) | 2 (33.3%) | 4 (57.1%) | 12 (42.3%) |
| Asian | 0 (0%) | 0 (0%) | 1 (16.7%) | 0 (0%) | 1 (3.8%) |
| [symbol] | | | | | |
| dsDNA + | 3 (60%) | 7 (87.5%) | 5 (83.3%) | 5 (71.4%) | 20 (76.9%) |
| Clinical Involvement | | | | | |
| Renal | 2 (40%) | 4 (50%) | 0 (0%) | 4 (57.1%) | 10 (38.3%) |
| Neurological | 0 (0%) | 0 (0%) | 1 (16.7%) | 2 (28.0%) | 3 (31.3%) |
| Fatigue | 2 (40%) | 8 (75%) | 5 (83.3%) | 2 (28.0%) | 15 (57.7%) |
| Clinical Response | | | | | |
| 3M Depletion | 4 (80%) | 3 (20%) | 4 (58.7%) | 4 (37.1%) | 15 (62.5%) |
| | | [symbol] | | | [symbol] |
| 6M BILAG Response | | | | | |
| Complete/Partial | 4 (80%) | 2 (25%) | 2 (33.3%) | 5 (21.4%) | 12 (42.3%) |
| No | 1 (20%) | 4 (15%) | 4 (60.7%) | 2 (28.6%) | 12 (48.2%) |

M—Months;
Depletion defined as less than 5 CD19+ B cells/μl [symbol]
indicates data missing or illegible when filed

Serum sCD23 and BAFF were weakly correlated in all samples (r2=0.11; p=0.001). Levels of both analytes from individual patients were found to follow broadly consistent patterns. Patients could therefore be grouped according to levels greater or lower than ULN: Group I—High sCD23, Low BAFF; Group II—High sCD23, High BAFF; Group III—Low sCD23, Low BAFF; Group IV—Low sCD23, High BAFF. Patients in Groups I and IV had higher BILAG responses at 6 months. No patients in Group III had renal involvement.

### Example 2

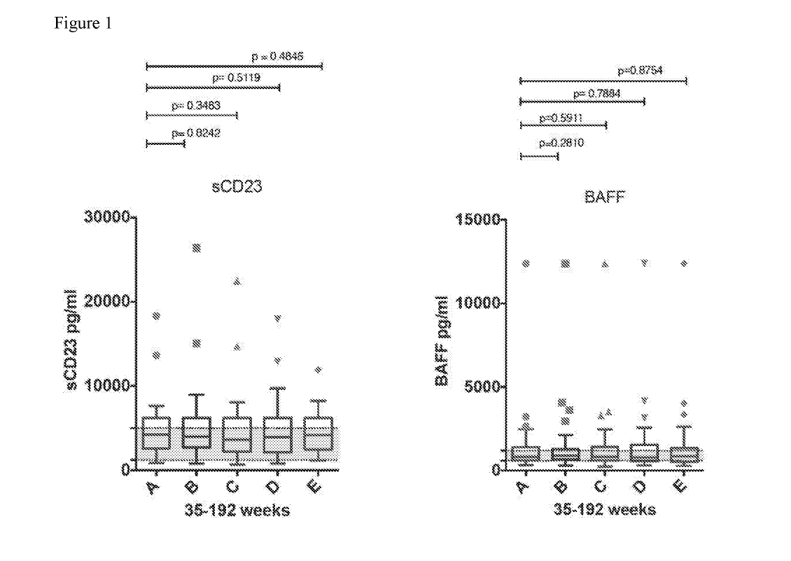

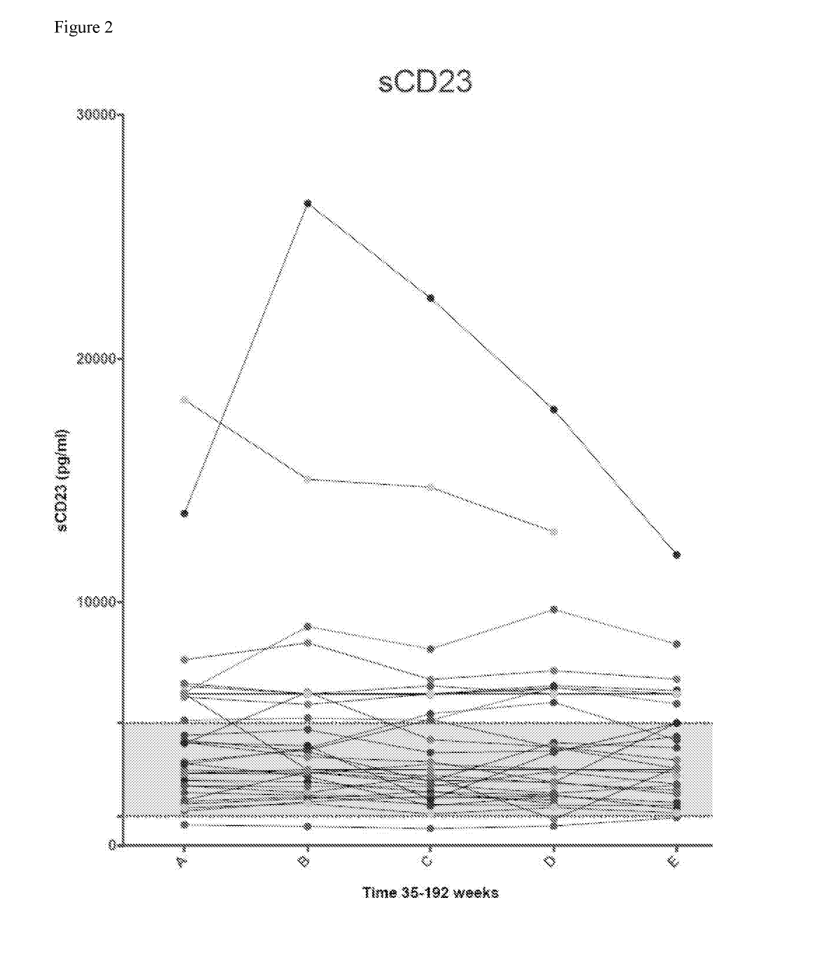

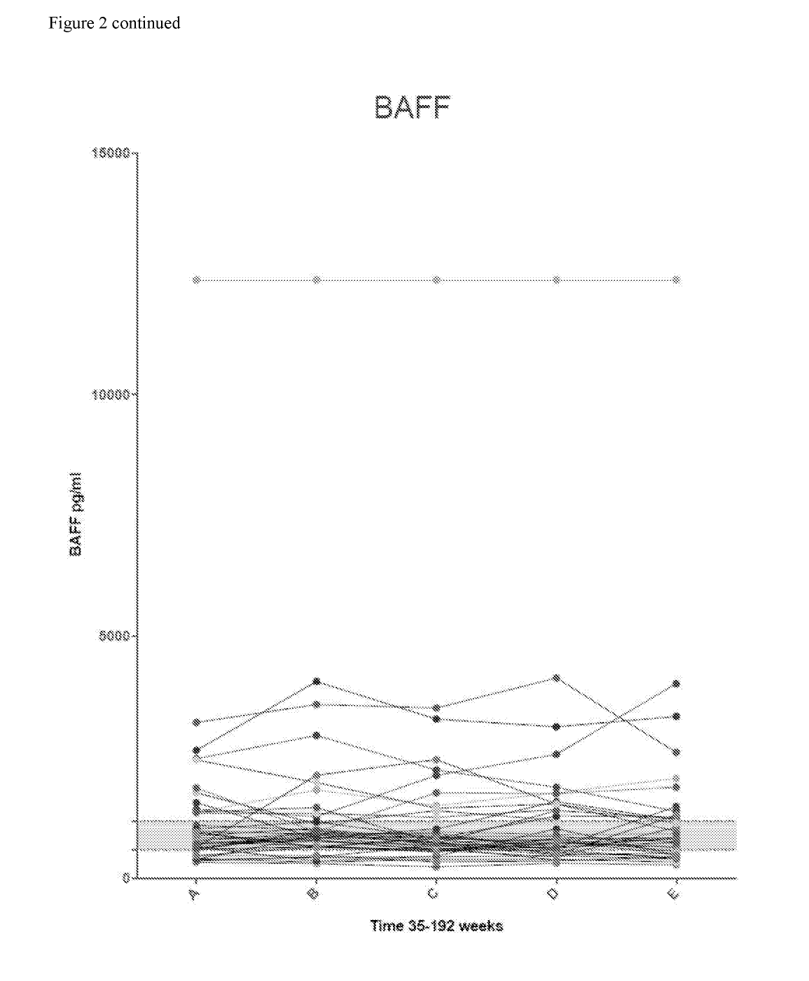

The level of BAFF and soluble CD23 biomarkers was analysed over a period of time using the same techniques as above. FIG. 1 shows summary data for 39 consecutive patients with a diagnosis of SLE (ACR Guidelines) referred to UCLH, and from whom at least 4 serial serum samples were available within a 24 month period. All patients were Rituximab naïve. Times between samples varied and therefore data was grouped in terms of order of sample collection which was at least 2-3 months apart in each patient. Samples were tested for sCD23 and BAFF. Data were grouped for each respective time point (A being first, B being second etc). p values were calculated using non-parametric statistics (Wilcoxon test for paired samples) in order to assess relative change between variables. (FIG. 2 shows individual subject data.)

The results shown in FIGS. 1 and 2 demonstrate that the relative levels of BAFF and sCD23 remain remarkably constant over time (up to 2 years) in rituximab-naïve patients with SLE. This is despite the well-described fluctuations in disease activity and changing manifestations of disease over time in SLE patients and also the administration of therapeutic agents. This suggests that the relationship between BAFF and sCD23 reflect underlying mechanisms of disease.

1. A method for predicting the response of a subject with an autoimmune disease to a B cell-targeting therapy, wherein said method comprises analysing the level of soluble CD23 and B cell activation factor (BAFF) in a sample from said subject.

2. The method according to claim 1, further comprising the step of:
comparing the level of said soluble CD23 and BAFF in the sample to a reference value, wherein the level of soluble CD23 and BAFF in the sample compared to the reference value is predictive of the subject's response to a B cell-targeting therapy.

3. The method according to claim 1 or 2 wherein a higher level of soluble CD23 and a lower level of BAFF is predictive of an improved response to a B cell-targeting therapy.

4. The method according to claim 1 or 2 wherein a higher level of BAFF and a lower level of soluble CD23 is predictive of an improved response to a B cell-targeting therapy.

5. The method according to claim 1 or 2 wherein a higher level of soluble CD23 and a higher level of BAFF is predictive of a poorer response to a B cell-targeting therapy.

6. The method according to claim 1 or 2 wherein a lower level of soluble CD23 and a lower level of BAFF is predictive of a poorer response to a B cell-targeting therapy.

7. Use of soluble CD23 and BAFF as biomarkers for predicting the response of a subject with an autoimmune disease to a B cell-targeting therapy.

8. The method or use according to any preceding claim wherein said method or use identifies a subject suitable for treatment with a B cell-targeting therapy.

9. The method or use according to claim 8 wherein said autoimmune disease is systemic lupus erythematosus.

10. The method or use according to any preceding claim comprising determining the polypeptide level of soluble CD23 and BAFF.

11. The method or use according to claim 10, wherein the polypeptide level is determined by ELISA.

12. The method or use according to any preceding claim, wherein the sample is a blood sample.

13. The method or use according to claim 12, wherein the sample is a blood serum sample.

14. The method or use according to any preceding claim, wherein the subject is human.

15. The method or use according to any preceding claim, wherein the B cell-targeting therapy is an anti-CD20 antibody.

16. The method or use according to claim 15, wherein the B cell-targeting therapy is rituximab.

17. A method of treating an autoimmune disease in a subject predicted to have an improved response to a B cell-targeting therapy by the method according to any preceding claim, comprising administering a therapeutically effective amount of a B cell-targeting therapy to the subject.

18. A composition comprising a therapeutically effective amount of a B cell-targeting therapy for use in the treatment of an autoimmune disease in a subject predicted to have an improved response to a B cell-targeting therapy by the method according to any preceding claim.

19. The method according to claim 17 or composition according to claim 18, wherein the B cell-targeting therapy is rituximab.

20. The method according to claim 17 or 19, or composition according to claim 18 or 19, wherein the autoimmune disease is systemic lupus erythematosus.

21. A kit for predicting the response to a B cell-targeting therapy of a subject with an autoimmune disease, wherein the kit comprises one or more agents capable of determining the level of soluble CD23 and BAFF in a sample from the subject, wherein the kit optionally comprises a set of instructions.

22. Use of a kit according to claim 21 for predicting the response to a B cell-targeting therapy of a subject with an autoimmune disease.

23. A method, use, composition for use or kit substantially as described herein and with reference to the accompanying Examples.
